(19) United States
(12) Patent Application Publication
(10) Pub. No.: US 2023/0407282 A1
(43) Pub. Date: Dec. 21, 2023

(54) SYSTEMS AND METHODS FOR TREATING CONDITIONS AND DISEASES USING ALTERNATING ELECTRIC FIELDS AND CRISPR-CAS SYSTEM

(71) Applicant: Novocure GmbH, Root D4 (CH)

(72) Inventors: Tal KAN, Haifa (IL); Lilach AVIGDOR, Haifa (IL); Tali VOLOSHIN-SELA, Haifa (IL)

(73) Assignee: Novocure GmbH, Root D4 (CH)

(21) Appl. No.: 18/336,493

(22) Filed: Jun. 16, 2023

Related U.S. Application Data
(60) Provisional application No. 63/353,946, filed on Jun. 21, 2022.

(51) Int. Cl. C12N 9/22; A61N 1/36

(52) U.S. Cl. CPC C12N 9/22; A61N 1/36002; A61N 1/36171; C12N 2310/20

(57) ABSTRACT

Systems and methods are disclosed for inducing apoptosis and treating or reducing the occurrence of at least one condition, disease, disorder, or infection in a subject. The systems and methods rely on the application of an alternating electric field and administration of a clustered regularly interspaced short palindromic repeats (CRISPR) associated protein (CRISPR-Cas) system. Also provided are kits for performing the methods disclosed herein.

### CROSS REFERENCE TO RELATED APPLICATIONS/INCORPORATION BY REFERENCE STATEMENT

The subject application claims benefit under 35 USC § 119(e) of U.S. provisional application No. 63/353,946, filed Jun. 21, 2022. The entire contents of the above-referenced patent application(s) are hereby expressly incorporated herein by reference.

### STATEMENT REGARDING FEDERALLY SPONSORED RESEARCH OR DEVELOPMENT

Not Applicable.

### BACKGROUND

Tumor Treating Fields (TTFields) are low intensity (e.g., 1-3 V/cm) alternating electric fields within the intermediate frequency range (100-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the electrode arrays that make up each of these pairs are positioned on opposite sides of the body part that is being treated. More specifically, for the OPTUNE® system, one pair of electrodes is located to the left and right (LR) of the tumor, and the other pair of electrodes is located anterior and posterior (AP) to the tumor. TTFields are approved for the treatment of glioblastoma multiforme (GBM), and may be delivered, for example, via the OPTUNE® system (Novocure Limited, St. Helier, Jersey), which includes transducer arrays placed on the patient's shaved head.

Each transducer array used for the delivery of TTFields in the OPTUNE® device comprises a set of ceramic disk electrodes, which are coupled to the patient's skin (such as, but not limited to, the patient's shaved head for treatment of GBM) through a layer of conductive medical gel. The purpose of the medical gel is to deform to match the body's contours and to provide good electrical contact between the arrays and the skin; as such, the gel interface bridges the skin and reduces interference. The device is intended to be continuously worn by the patient for 2-4 days before removal for hygienic care and re-shaving (if necessary), followed by reapplication with a new set of arrays. As such, the medical gel remains in substantially continuous contact with an area of the patient's skin for a period of 2-4 days at a time, and there is only a brief period of time in which the area of skin is uncovered and exposed to the environment before more medical gel is applied thereto.

TTFields have been shown to delay DNA damage repair mechanisms, and specifically the mechanisms responsible for repairing double strand DNA breaks; in particular, but not by way of limitation, BRCA1 and Fanconi anemia proteins have been shown to be downregulated in gene and protein expression following application of alternating electric fields thereto (Giladi et al. (2017) Radiation Oncology, 12(1):206; Karanam et al. (2017) Cell Death Dis, 8(3):e2711; Mumblat et al. (2021) International Journal of Radiation Oncology, Biology, Physics, 111(3):e463).

Clustered regularly interspaced short palindromic repeat-CRISPR associated nuclease protein—known as CRISPR-Cas9—is a system for gene editing derived from prokaryotes that is a key component of their adaptive immunity. This gene editing mechanism of the CRISPR-Cas9 system has been adapted and used in mammalian cells to silence or promote the expression of genes in precise sequences in the DNA. The mechanism in which CRISPR-Cas9 works is by recognizing DNA elements with a single guide RNA against a specific sequence of interest, which is then cut by the Cas9 enzyme in double strand DNA nicks (Zhang et al. (2021) Mol. Cancer, 20:126; Afolabi et al. (2019) Immunology, 158(2):63-69). At this point, DNA repair mechanisms are employed in the current uses of the CRISPR-Cas9 system (Richardson et al. (2018) Nature Genetics, 50(8):1132-1139).

However, there is a need in the art for new and improved systems and methods for treating or reducing the occurrence of cancer and other conditions and diseases. It is to such new and improved systems and methods that the present disclosure is directed.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “single guide RNA” or “sgRNA” refer to a DNA-targeting RNA containing a guide sequence that targets a CRISPR-associated endonuclease to the target genomic DNA and a scaffold sequence that interacts with the endonuclease (e.g., tracrRNA).

The terms “CRISPR-associated endonuclease,” “Cas polypeptide,” and “Cas nuclease” refer to a family of Clustered Regularly Interspaced Short Palindromic Repeats-associated polypeptides or nucleases that cleave DNA to generate blunt ends at the double-strand break at sites specified by a nucleotide guide sequence contained within a crRNA transcript. A CRISPR-associated endonuclease requires both a crRNA and a tracrRNA for site-specific DNA recognition and cleavage. The crRNA associates, through a region of partial complementarity, with the tracrRNA to guide the endonuclease to a region homologous to the crRNA in the target DNA called a “protospacer.”

The term “ribonucleoprotein complex” or “RNP complex” refers to a complex comprising an sgRNA and a CRISPR-associated endonuclease polypeptide.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “patient” or “subject” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

The term “therapeutic composition” or “pharmaceutical composition” as used herein refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof, or an amount of a treatment protocol (i.e., an alternating electric field), sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one condition, disease, and/or infection. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated with or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

Turning now to the inventive concept(s), systems, kits, and methods are provided for inducing cellular apoptosis and/or treating or reducing the occurrence of at least one condition, disease, disorder, or infection in a subject. The systems, kits, and methods combine the application of alternating electric fields with the clustered regularly interspaced short palindromic repeats (CRISPR) associated protein (CRISPR-Cas) system. Alternating electric fields (such as, but not limited to, TTFields) delay DNA damage repair mechanisms, and specifically the mechanisms responsible for repairing double strand DNA breaks. The CRISPR-Cas is a system for gene editing that has been adapted and used in mammalian cells to silence or promote the expression of genes in precise sequences in the DNA. The CRISPR-Cas9 system recognizes DNA elements with a single guide RNA (sgRNA) against a specific sequence of interest, which is then cut by the Cas9 enzyme in double strand DNA nicks. Currently, the CRISPR-Cas9 system then employs DNA repair mechanisms. However, in the present disclosure, since application of the alternating electric fields inhibits DNA damage repair mechanisms, the DNA nicks formed upon exposure to the CRISPR-Cas system will not be repaired; as such, the cell(s) exposed to the combined treatment of alternating electric fields with the CRISPR-Cas system will undergo apoptosis.

This ability to induce apoptosis efficiently only in mutated cells (or other target cells of interest that uniquely possess a specific sequence to which a sgRNA can be directed) offers patients a treatment option with less toxicities and/or adverse events compared to systemic therapies such as (but not limited to) chemotherapy.

Certain non-limiting embodiments of the present disclosure include methods for inducing apoptosis in at least one cell. In the method, an alternating electric field is applied to the at least one cell for a period of time, and application of the alternating electric field downregulates at least one DNA damage repair pathway in the at least one cell. The at least one cell is also exposed to a clustered regularly interspaced short palindromic repeats (CRISPR) associated protein (CRISPR-Cas) system that includes (i) a single guide RNA (sgRNA) that comprises a guide sequence that hybridizes to a target DNA sequence in the at least one cell, and (ii) a CRISPR-associated endonuclease or gene encoding same.

Certain non-limiting embodiments of the present disclosure include methods of treating or reducing the occurrence of at least one condition, disease, disorder, or infection in a subject. In the method, an alternating electric field is applied to at least a portion of the subject for a period of time, and application of the alternating electric field downregulates at least one DNA damage repair pathway in at least a portion of the subject. A CRISPR-Cas system is also administered to the subject, and the CRISPR-Cas system includes (i) a single guide RNA (sgRNA) that comprises a guide sequence that hybridizes to a target DNA sequence associated with the at least one condition, disease, disorder, or infection; and (ii) a CRISPR-associated endonuclease or gene encoding same.

Any type of conductive or non-conductive electrode(s) and/or transducer array(s) that can be utilized for generating an alternating electric field that are known in the art or otherwise contemplated herein may be applied to the cell(s)/subject for generation of the alternating electric field in accordance with the present disclosure. Non-limiting examples of electrodes and transducer arrays that can be utilized for generating an alternating electric field in accordance with the present disclosure include those that function as part of a TTFields system as described, for example but not by way of limitation, in U.S. Pat. Nos. 7,016,725; 7,089,054; 7,333,852; 7,565,205; 8,244,345; 8,715,203; 8,764,675; 10,188,851; 10,441,776; and 11,452,863; and in US Patent Application Nos. US 2018/0001078; US 2018/0160933; US 2019/0117956; US 2019/0307781; and US 2019/0308016.

The alternating electric field may be generated at any frequency that downregulates at least one DNA damage repair pathway in at least a portion of the cell(s)/subject, in accordance with the present disclosure. For example (but not by way of limitation), the alternating electric field may have a frequency of about 50 kHz, about 75 kHz, about 100 kHz, about 125 kHz, about 150 kHz, about 175 kHz, about 200 kHz, about 225 kHz, about 250 kHz, about 275 kHz, about 300 kHz, about 325 kHz, about 350 kHz, about 375 kHz, about 400 kHz, about 425 kHz, about 450 kHz, about 475 kHz, about 500 kHz, about 550 kHz, about 600 kHz, about 650 kHz, about 700 kHz, about 750 kHz, about 800 kHz, about 850 kHz, about 900 kHz, about 950 kHz, about 1 MHz, about 2 MHz, about 3 MHz, about 4 MHz, about 5 MHz, about 6 MHz, about 7 MHz, about 8 MHz, about 9 MHz, about 10 MHz, about 11 MHz, about 12 MHz, about 13 MHz, about 14 MHz, about 15 MHz, and the like, as well as a range formed from any of the above values (e.g., a range of from about 100 kHz to about 10 MHz, a range of from about 50 kHz to about 1 MHz, a range of from about 1 MHz to about 10 MHz, a range of from about 50 kHz to about 500 kHz, a range of from about 100 kHz to about 300 kHz, a range of from about 150 kHz to about 300 kHz, etc.), and a range that combines two integers that fall between two of the above-referenced values (e.g., a range of from about 122 kHz to about 313 kHz, a range of from about 78 kHz to about 298 kHz, etc.).

In certain particular (but non-limiting) embodiments, the alternating electric field may be imposed at two or more different frequencies, either simultaneously or sequentially during treatment (i.e., the frequency may be changed during treatment). When two or more frequencies are present, each frequency is selected from any of the above-referenced values, or a range formed from any of the above-referenced values, or a range that combines two integers that fall between two of the above-referenced values.

The alternating electric field may have any field strength so long as the alternating electric field is capable of downregulating at least one DNA damage repair pathway in at least a portion of the cell(s)/subject, in accordance with the present disclosure. For example (but not by way of limitation), the alternating electric field may have a field strength of at least about 0.1 V/cm, about 0.5 V/cm, about 1 V/cm, about 1.5 V/cm, about 2 V/cm, about 2.5 V/cm, about 3 V/cm, about 3.5 V/cm, about 4 V/cm, about 4.5 V/cm, about 5 V/cm, about 5.5 V/cm, about 6 V/cm, about 6.5 V/cm, about 7 V/cm, about 7.5 V/cm, about 8 V/cm, about 9 V/cm, about 9.5 V/cm, about 10 V/cm, about 10.5 V/cm, about 11 V/cm, about 11.5 V/cm, about 12 V/cm, about 12.5 V/cm, about 13 V/cm, about 13.5 V/cm, about 14 V/cm, about 14.5 V/cm, about 15 V/cm, about 15.5 V/cm, about 16 V/cm, about 16.5 V/cm, about 17 V/cm, about 17.5 V/cm, about 18 V/cm, about 18.5 V/cm, about 19 V/cm, about 19.5 V/cm, about 20 V/cm, and the like, as well as a range formed from any of the above values (e.g., a range of from about 1 V/cm to about 20 V/cm, a range of from about 1 V/cm to about 10 V/cm, a range of from about 1 V/cm to about 4 V/cm, etc.), and a range that combines two integers that fall between two of the above-referenced values (e.g., a range of from about 1.1 V/cm to about 18.6 V/cm, a range of from about 1.2 V/cm to about 9.8 V/cm, a range of from about 1.3 V/cm to about 4.7 V/cm, etc.).

The alternating electric field may be applied for any period of time sufficient to downregulate at least one DNA damage repair pathway in at least a portion of the cell(s)/subject. For example, but not by way of limitation, the alternating electric field may be applied for at least about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, as well as a range formed from any of the above values (e.g., a range of from about 24 hours to about 72 hours, etc.), and a range that combines two integers that fall between two of the above-referenced values (e.g., a range of from about 14 hours to about 68 hours, etc.).

The total period of time that the alternating electric field is applied may be achieved in a continuous or intermittent manner. That is, when the alternating electric field is applied for a shorter period of time (such as, but not limited to, less than about 12 or 24 hours), the alternating electric field may be continuously applied over that period of time. However, when the alternating electric field is applied for a longer period of time (such as, but not limited to, a period of about 24 hours or greater), the treatment period may include one or more breaks during the application cycle that separate two or more application sections, whereby the application sections and breaks combine to form the total application period. When breaks are present, the breaks should typically constitute about 50%, about 40%, about 30%, about 20% or less of the treatment time, so that the alternating electric field is applied for at least about 50%, about 60%, about 70%, about 80% or more of the treatment time. For example, but not by way of limitation, the alternating electric field should be applied for at least about 19 hours of each 24-hour period. In addition, the longer that the alternating electric field is applied, the higher the efficacy will be.

Any CRISPR-Cas system known in the art or otherwise contemplated herein may be utilized in accordance with the present disclosure, so long as the system is capable of creating double strand DNA nicks at a target sequence of interest. CRISPR-Cas systems are widely known and have been extensively reviewed (see, for example, Adli, M. (2018) Nat Commun, 9:1911; Pickar-Oliver et al. (2019) Nature Reviews Molecular Cell Biology, 20:490-507; Xu et al. (2020) Comput Struct Biotechnol J., 18:2401-2415; Nidhi et al. (2021) Int J Mol Sci., 22(7):3327). In addition, CRISPR-Cas systems are commercially available from a wide variety of sources; see, for example, Merck/MilliporeSigma (Burlington, MA), Takara Bio USA, Inc. (San Jose, CA), ThermoFisher Scientific (Waltham, MA), Integrated DNA Technologies, Inc. (Coralville, IA), GenScript Biotech Corp (Piscataway, NJ), ACROBiosystems (Newark, DE), Mirus Bio LLC (Madison, WI), and the like.

The CRISPR-Cas system will include any CRISPR-associated endonuclease or gene encoding same that is known in the art or otherwise contemplated herein for use within a CRISPR-Cas system to recognize and cleave target DNAs with complementarity to the guide RNA. Non-limiting examples of Cas endonucleases that may be utilized in accordance with the present disclosure include Cas3, Cas9, Cas12, Cas12a, Cas12b, Cas12e, Cas12f, Cas13a, Cas13b, mini-Cas9, Cas9 nickase (nCas9), and the like, as well as variants and derivatives thereof, and any combinations thereof.

Depending on the particular Cas endonuclease utilized, the sgRNA of the CRISPR-Cas system will contain sequence(s) specific to the particular Cas endonuclease that are recognized by that endonuclease and that direct the endonuclease to make sequence-specific cuts in the target DNA sequence. Selection and/or creation of a sgRNA that contains these endonuclease-directing sequence(s) in combination with sequence(s) that hybridize to the target DNA sequence is well within the purview of a person of ordinary skill in the art, and therefore no further description thereof is deemed necessary.

In certain non-limiting embodiments, the methods of the present disclosure can be applied to various conditions in which a mutation occurs and results in silencing or over activation of a damaged gene, or to conditions that involve foreign genes/sequences. For example (but not by way of limitation), the at least one condition, disease, disorder, or infection is selected from the group consisting of a cancer, an autoimmune disease, a viral infection, a bacterial infection, a fungal infection, a parasitic infection, a metabolic syndrome, Huntington's disease, Von Hippel-Lindau syndrome, and the like, as well as any combinations thereof.

Particular non-limiting examples of conditions, diseases, disorders, and infections that can be treated in accordance with the present disclosure include:
cancers that involve an oncological gain of function gene mutation(s) in an oncogene (see, for example (but not by way of limitation) Kontomanolis et al. (2020) Anticancer Research, 40(11):6009-6015; Frank et al. (1998) Journal of Clinical Oncology 16:7:2417-2425) and/or a loss of function mutation(s) in a tumor suppressor gene (see, for example (but not by way of limitation) Chen et al. (2020) Sig Transduct Target Ther, 5:90);
autoimmune diseases, in which the sgRNA of the CRISPR-Cas system is directed to specific sequences of self-antigens, thereby killing immune cells that are improperly directed against “self” cells (i.e., immune cells that produce autoantibodies);
infections, where the sgRNA of the CRISPR-Cas system is directed against one or more genes/sequences from the infectious microorganism (such as, but not limited to, a bacteria, parasite, fungus, orvirus)—see, for example, Hu et al. (2014) BioMed Research International, Article ID 612823. In addition, a CRISPR-Cas9 system has been utilized to target the LTR regions of the HIV provirus at positions T5 and T6 (Ebina et al. (2013) Sci Rep, 3:2510);
metabolic syndromes such as (but not limited to) hereditary tyrosinemia (Pankowicz et al. (2016) Nat Commun, 7:12642), where the sgRNA of the CRISPR-Cas system targets mutations in (for example, but not by way of limitation) the FAH, TAT, and HPD genes that cause tyrosinemia types I, II, and III, respectively;
Huntington's disease, where the sgRNA of the CRISPR-Cas system targets the CAG repeat expansion, as Huntington's disease is characterized by a loss of brain striatal neurons that occurs as a consequence of an expansion of a CAG repeat in the huntingtin protein (see, for example (but not by way of limitation) Cattaneo et al., (2001) Trends in Neurosciences, 24(3):182-188).

In particular (but non-limiting) embodiments, the target DNA sequence is directed to a mutation in a gene selected from the group consisting of p53, retinoblastoma (Rb), phosphatase and tensin homolog deleted on chromosome ten (PTEN), Ras association domain family (RASSF), ADP-ribosylation factor (ARF), adenomatous polyposis coli (APC), ataxia telangiectasia mutated (ATM), checkpoint kinase 2 (CHK2), breast cancer 1 protein (BRCA1), breast cancer 2 protein (BRCA2), partner and locator of BRCA2 (PALB2), tuberous sclerosis complex 1 (TSC1), tuberous sclerosis complex 2 (TSC2), neurofibromatosis type 1 (NF1), liver kinase B1 (LKB1), E2F1, KLF5, forkhead box class 03a (FOXO3a), Ras (H-Ras, K-Ras, and N-Ras), Mitogen-activated protein kinase kinase (MEK), extracellular signal-regulated kinase (ERK), c-Jun NH2-terminal kinase (JNK), p38, human epidermal growth factor receptor 2 (HER2), epidermal growth factor receptor (EGFR), c-Myc, Von Hippel-Lindau (VHL), fumarylacetoacetate hydrolase (FAH), TAT, 4-hydroxyphenylpyruvate dioxygenase (HPD), fms-related tyrosine kinase 3 (FLT3), c-KIT, BRAF, phosphatidylinositol-4,5-bisphosphate 3-kinase (PIK3CA), telomerase reverse transcriptase (TERT), ETS variant transcription factor 1 (ETV1), β-catenin (CTNNB1), E2F1, T cell acute lymphocytic leukemia 1 (TAL1), and the like, as well as combinations thereof.

In certain particular (but non-limiting) embodiments, the target DNA sequence is a loss of function mutation in a tumor suppressor gene; examples of tumor suppressor genes include (but are not limited to) p53, retinoblastoma (Rb), phosphatase and tensin homolog deleted on chromosome ten (PTEN), Ras association domain family (RASSF), ADP-ribosylation factor (ARF), adenomatous polyposis coli (APC), ataxia telangiectasia mutated (ATM), checkpoint kinase 2 (CHK2), breast cancer 1 protein (BRCA1), breast cancer 2 protein (BRCA2), partner and locator of BRCA2 (PALB2), tuberous sclerosis complex 1 (TSC1), tuberous sclerosis complex 2 (TSC2), neurofibromatosis type 1(NF1), liver kinase B1(LKB1), E2F1, KLF5, forkhead box class 03a (FOXO3a), p38, Von Hippel-Lindau (VHL), and the like, as well as any combinations thereof.

In certain particular (but non-limiting) embodiments, the target DNA sequence is an oncological gain of function gene mutation (i.e., oncogene); examples of oncogenes include (but are not limited to) Ras (H-Ras, K-Ras, and N-Ras), Mitogen-activated protein kinase kinase (MEK), extracellular signal-regulated kinase (ERK), c-Jun NH2-terminal kinase (JNK), human epidermal growth factor receptor 2 (HER2), epidermal growth factor receptor (EGFR), c-Myc, fumarylacetoacetate hydrolase (FAH), TAT, 4-hydroxyphenylpyruvate dioxygenase (HPD), fms-related tyrosine kinase 3 (FLT3), c-KIT, BRAF, phosphatidylinositol-4,5-bisphosphate 3-kinase (PIK3CA), telomerase reverse transcriptase (TERT), ETS variant transcription factor 1 (ETV1), 3-catenin (CTNNB1), E2F1, T cell acute lymphocytic leukemia 1 (TAL1), and the like, as well as any combinations thereof.

In certain particular (but non-limiting) embodiments, the target DNA sequence is a gene from a microorganism selected from the group consisting of Human papillomavirus (HPV), Hepatitis B and C viruses (HBV and HCV, respectively), Epstein Barr virus (EBV), human immunodeficiency virus (HIV), SARS-CoV-2 virus, Herpes Simplex virus (HSV), Human Herpes Virus 8 (HHV-8), Human T-lymphotropic virus-1 (HTLV-1), Merkel Cell Polyomavirus (MCPyV), Cytomegalovirus (CMV), Helicobacter pylori, Chlamydia trachomatis, Salmonella typhi, Salmonella paratyphi A, Salmonella typhimurium, Streptococcus bovis, Mycoplasma, and the like, as well as any combinations thereof.

The CRISPR-Cas system cargo may be administered to the cells/subject in any form, via any delivery vehicle(s), and via any delivery mechanism(s)/route(s) known or otherwise contemplated herein for delivery of the CRISPR-Cas system cargo, so long as the form and delivery vehicle(s) are capable of transporting the CRISPR-Cas system into the cell(s) of interest and facilitating penetration into the nucleus so as to facilitate the creation of double strand DNA nicks at a target sequence of interest. The CRISPR-associated endonuclease or gene encoding same may be present in the form of a protein, peptide, DNA molecule, RNA molecule (such as, but not limited to, a mRNA molecule), or any combination thereof, depending on the delivery vehicle used.

The sgRNA and CRISPR-associated endonuclease (or gene encoding same) may be delivered via a single delivery vehicle or may be separated between two different delivery vehicles (which may be the same or different types of vehicles). In certain non-limiting embodiments, the CRISPR-Cas system may be delivered as a ribonucleoprotein (RNP) complex formed of sgRNA and endonuclease peptide or protein. In certain non-limiting embodiments, one or both components of the CRISPR-Cas system may be delivered via one or more DNA plasmids containing sequence(s) of the sgRNA and/or sequence(s) encoding the endonuclease. In other non-limiting embodiments, one or both components of the CRISPR-Cas system may be delivered via one or more DNA- or RNA-based viral vectors (such as, but not limited to, an adenoviral, adeno-associated viral, lentiviral, retroviral, vaccinia, and/or herpes simplex viral vector, and the like) or an artificial virus. In other non-limiting embodiments, one or both components of the CRISPR-Cas system may be delivered via liposomes and/or lipid nanoparticles; immunoliposomes; lipoplexes, polyplexes, or poloxamers; polycations; virosomes; gold nanoparticles or other inorganic nanoparticles; and the like. In yet other non-limiting embodiments, one or both components of the CRISPR-Cas system are delivered in naked form via straight physical delivery. In yet further non-limiting embodiments, the sgRNA is delivered via one of the above vehicles, while the CRISPR-associated endonuclease (or nucleic acid sequence encoding same) is delivered via another of the above vehicles.

In certain non-limiting embodiments, one or both components of the CRISPR-Cas system and/or the delivery vehicle may be modified, such as (but not by way of limitation) to further aid in transporting the CRISPR-Cas system into the cancer cell, enhancing solubility, monitoring transport, and the like. For example (but not by way of limitation), the sgRNA, endonuclease (or gene encoding same), and/or delivery vehicle may be engineered to comprise a nuclear localization sequence (NLS), a cell penetrating peptide or peptide sequence, an affinity tag, a detectable tag (such as, but not limited to, a fluorescent tag), a cancer cell-targeting ligand or receptor, and the like, as well as any combination thereof.

The components of the CRISPR-Cas system may be delivered by any delivery mechanisms/routes known in the art or otherwise contemplated herein. Non-limiting examples of delivery mechanisms/routes include injection, infusion, topical application, implantation, electroporation, lipofection, microinjection, microfluidics, biolistics, particle gun acceleration, and the like, as well as any combinations thereof.

The alternating electric fields may be applied to any portion(s) of a subject that would result in downregulation of at least one DNA damage repair pathway in a desired portion of the cell(s)/subject. In certain particular (but non-limiting) embodiments, the alternating electric fields are targeted to a portion of the subject containing the gene or gene mutation to be targeted. For example (but not by way of limitation), when at least one solid tumor is present, the alternating electric fields may be applied to a portion of the subject containing the solid tumor. Alternatively, when the treatment is targeting an infection in the subject, the alternating electric fields may be applied to at least a portion of the infection site on or in the subject (such as, but not limited to, the cervix for HPV infection, the thymus or infection site for HIV infection and AIDS-induced conditions (such as, but not limited to HIV-induced pneumonia). In yet other alternatives, when the treatment is targeting an autoimmune disease, the alternating electric fields may be applied to the inflammation target's draining lymph node; when the treatment is targeting a metabolic syndrome such as (but not limited to) hereditary tyrosinemia, the alternating electric fields may be applied to the liver; and when the treatment is targeting Huntington's disease, the alternating electric fields may be applied to the brain.

The methods of the present disclosure may include one or more additional steps. For example, but not by way of limitation, the methods may include one or more steps that aid in the identification of subjects to be treated, one or more additional administration steps (such as, but not limited to, the administration of at least one substance for concurrent therapies), and/or one or more steps for analyzing the effectiveness of the treatment.

In one non-limiting embodiment, the method further includes the step of examining a sample and identifying the target DNA sequence in the sample. This step may be performed before and/or after one or both of the alternating electric field application and CRISPR-Cas system administration steps.

In certain particular (but non-limiting) embodiments, the CRISPR-Cas system may be administered after the application of the alternating electric field has begun. In particular (but not by way of limitation), the CRISPR-Cas system may be administered during application of the alternating electric field and/or after application of the alternating electric field has elapsed.

For example (but not by way of limitation), the CRISPR-Cas system may be administered after application of the alternating electric field has commenced by a period of at least about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, as well as a range formed from any of the above values (e.g., a range of from about 24 hours to about 96 hours, a range of from about 24 hours to about 48 hours, etc.), and a range that combines two integers that fall between two of the above-referenced values (e.g., a range of from about 14 hours to about 94 hours, etc.). In a particular (but non-limiting) embodiment, the at least one substance is orally administered at least about 24 hours after application of the alternating electric field has begun.

In other non-limiting examples, CRISPR-Cas system may be administered after the period of time that the alternating electric field is applied has elapsed, wherein the at least one substance is administered within about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, of when the period of time elapsed. In a particular (but non-limiting) embodiment, the CRISPR-Cas system is administered within about 96 hours of when the period of time elapsed.

In certain particular (but non-limiting) embodiments, the method includes one or more additional steps. For example (but not by way of limitation), the method may further include the step of discontinuing the application of the alternating electric field (such as, but not limited to) to allow the cells/tissue to recover. In addition, any of the steps may be repeated one or more times. Each of the steps can be repeated as many times as necessary. When steps involving application of the alternating electric fields are repeated, the transducer arrays may be placed in slightly different positions on the subject than their original placement; relocation of the arrays in this manner may further increase the effectiveness of the treatment and also minimize any adverse skin effects caused by the alternating electric fields. In addition, steps involving administration of the CRISPR-Cas system may be repeated various times and at various intervals to follow any known and/or generally accepted regimen for the system.

Certain non-limiting embodiments of the present disclosure are related to kits that include any of the components of the alternating electric field generating systems (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; 10,441,776; and 11,452,863; and in US Patent Application Nos. US 2018/0001078; US 2018/0160933; US 2019/0117956; US 2019/0307781; and US 2019/0308016) and one or more of any of the CRISPR-Cas systems disclosed or otherwise contemplated herein. In particular (but not by way of limitation), the kit may include (i) at least one pair of transducer arrays for generating an alternating electric field there between upon application of the transducer arrays to at least one cell and/or placement of the transducer arrays on a subject; and (ii) a CRISPR-Cas system comprising a single guide RNA (sgRNA) that comprises: a guide sequence that hybridizes to a target DNA sequence associated with the at least one condition, disease, disorder, or infection; and a CRISPR-associated endonuclease or gene encoding same. The condition, disease, disorder, or infection and the target DNA sequences may be any of those disclosed or otherwise contemplated herein.

In a particular (but non-limiting) embodiment, the kit may further include instructions for performing any of the methods disclosed or otherwise contemplated herein. For example (but not by way of limitation), the kit may include instructions for applying one or more components of an alternating electric field generating system to the patient's skin, instructions for applying the alternating electric field to a particular portion of the patient, instructions for when and how to administer the CRISPR-Cas system, and/or instructions for when to activate and turn off the alternating electric field in relation to the administration of the CRISPR-Cas system.

In addition to the components described in detail herein above, the kits may further contain other component(s)/reagent(s) for performing any of the particular methods described or otherwise contemplated herein. For example (but not byway of limitation), the kits may additionally include: (i) components for preparing the skin prior to disposal of the hydrogel compositions and/or transducer arrays thereon (e.g., a razor, a cleansing composition or wipe/towel, etc.); (ii) components for removal of the gel/transducer array(s); and/or (iii) components for cleansing of the skin after removal of the gel/transducer array(s). Alternatively (and/or in addition thereto), the kits may additionally at least one device and/or at least one reagent for examining a sample to identify if the target DNA sequence is present in the sample, and/or at least one device and/or at least one reagent for analyzing the effectiveness of the treatment protocol.

The nature of the various additional component(s)/reagent(s) that may optionally be present in the kits will depend upon the particular treatment format, and identification thereof is well within the skill of one of ordinary skill in the art; therefore, no further description thereof is deemed necessary.

The components/reagents present in the kits may each be in separate containers/compartments, or various components/reagents can be combined in one or more containers/compartments, depending on the sterility, cross-reactivity, and stability of the components/reagents.

The kit may be disposed in any packaging that allows the components present therein to function in accordance with the present disclosure. In certain non-limiting embodiments, the kit further comprises a sealed packaging in which the components are disposed. In certain particular (but non-limiting) embodiments, the sealed packaging is substantially impermeable to air and/or substantially impermeable to light.

In addition, the kit can further include a set of written instructions explaining how to use one or more components of the kit. A kit of this nature can be used in any of the methods described or otherwise contemplated herein.

In certain non-limiting embodiments, the kit has a shelf life of at least about six months, such as (but not limited to), at least about nine months, or at least about 12 months.

### NON-LIMITING ILLUSTRATIVE EMBODIMENTS OF THE INVENTIVE CONCEPT(S)

Illustrative embodiment 1. A method of inducing apoptosis in at least one cell, the method comprising the steps of: (1) applying an alternating electric field to the at least one cell for a period of time, wherein application of the alternating electric field downregulates at least one DNA damage repair pathway in the at least one cell; and (2) exposing the at least one cell to a clustered regularly interspaced short palindromic repeats (CRISPR) associated protein (CRISPR-Cas) system comprising: a single guide RNA (sgRNA) that comprises a guide sequence that hybridizes to a target DNA sequence in the at least one cell; and a CRISPR-associated endonuclease or gene encoding same.

Illustrative embodiment 2. The method of claim 1, wherein the CRISPR-associated endonuclease is selected from the group consisting of Cas3, Cas9, Cas12, Cas12a, Cas12b, Cas12e, Cas12f, Cas13a, Cas13b, mini-Cas9, and Cas9 nickase (nCas9).

Illustrative embodiment 3. The method of illustrative embodiment 1 or 2, wherein the target DNA sequence is selected from the group consisting of an oncological gain of function gene mutation in an oncogene, a loss of function mutation in a tumor suppressor gene, a gene of an infectious microorganism, and a self-antigen sequence in an immune cell that produces autoantibodies.

Illustrative embodiment 4. The method of illustrative embodiment 3, wherein the target DNA sequence is directed to a mutation in a gene selected from the group consisting of p53, retinoblastoma (Rb), phosphatase and tensin homolog deleted on chromosome ten (PTEN), Ras association domain family (RASSF), ADP-ribosylation factor (ARF), adenomatous polyposis coli (APC), ataxia telangiectasia mutated (ATM), checkpoint kinase 2 (CHK2), breast cancer 1 protein (BRCA1), breast cancer 2 protein (BRCA2), partner and locator of BRCA2 (PALB2), tuberous sclerosis complex 1 (TSC1), tuberous sclerosis complex 2 (TSC2), neurofibromatosis type 1 (NF1), liver kinase B1 (LKB1), E2F1, KLF5, forkhead box class 03a (FOXO3a), Ras (H-Ras, K-Ras, and N-Ras), Mitogen-activated protein kinase kinase (MEK), extracellular signal-regulated kinase (ERK), c-Jun NH2-terminal kinase (JNK), p38, human epidermal growth factor receptor 2 (HER2), epidermal growth factor receptor (EGFR), c-Myc, Von Hippel-Lindau (VHL), fumarylacetoacetate hydrolase (FAH), TAT, 4-hydroxyphenylpyruvate dioxygenase (HPD), fms-related tyrosine kinase 3 (FLT3), c-KIT, BRAF, phosphatidylinositol-4,5-bisphosphate 3-kinase (PIK3CA), telomerase reverse transcriptase (TERT), ETS variant transcription factor 1 (ETV1), 3-catenin (CTNNB1), E2F1, and T cell acute lymphocytic leukemia 1 (TAL1).

Illustrative embodiment 5. The method of illustrative embodiment 3, wherein the target DNA sequence is a gene from a microorganism selected from the group consisting of Human papillomavirus (HPV), Hepatitis B and C viruses (HBV and HCV, respectively), Epstein Barr virus (EBV), human immunodeficiency virus (HIV), SARS-CoV-2 virus, Herpes Simplex virus (HSV), Human Herpes Virus 8 (HHV-8), Human T-lymphotropic virus-1 (HTLV-1), Merkel Cell Polyomavirus (MCPyV), Cytomegalovirus (CMV), Helicobacter pylori, Chlamydia trachomatis, Salmonella typhi, Salmonella paratyphi A, Salmonella typhimurium, Streptococcus bovis, and Mycoplasma.

Illustrative embodiment 6. The method of any of illustrative embodiments 1-5, wherein the CRISPR-Cas system is administered in the form of a ribonucleoprotein (RNP) complex formed of sgRNA and endonuclease peptide or protein.

Illustrative embodiment 7. The method of any of illustrative embodiments 1-6, wherein at least one component of the CRISPR-Cas system is delivered via at least one DNA plasmid.

Illustrative embodiment 8. The method of any of illustrative embodiments 1-7, wherein at least one component of the CRISPR-Cas system is delivered via at least one DNA- or RNA-based viral vector.

Illustrative embodiment 9. The method of illustrative embodiment 8, wherein the viral vector is selected from the group consisting of an adenoviral, adeno-associated viral, lentiviral, retroviral, vaccinia, and/or herpes simplex viral vector, or an artificial virus.

Illustrative embodiment 10. The method of any of illustrative embodiments 1-9, wherein at least one component of the CRISPR-Cas system is delivered via at least one of a liposome, a lipid nanoparticle, an immunoliposome, a lipoplex, a polyplex, a poloxamer, a polycations, a virosome, a gold nanoparticle, other inorganic nanoparticle, and combinations thereof.

Illustrative embodiment 11. The method of any of illustrative embodiments 1-10, wherein at least one component of the CRISPR-Cas system is administered in naked form via straight physical delivery.

Illustrative embodiment 12. The method of any of illustrative embodiments 1-11, wherein the sgRNA and CRISPR-associated endonuclease or gene encoding same are administered by different delivery vehicles.

Illustrative embodiment 13. The method of any of illustrative embodiments 1-12, wherein at least one of the sgRNA and the gene encoding the CRISPR-associated endonuclease is modified to comprise a cell penetrating peptide sequence.

Illustrative embodiment 14. The method of any of illustrative embodiments 1-13, wherein at least one of the sgRNA and CRISPR-associated endonuclease or gene encoding same is administered by a route selected from the group consisting of injection, infusion, topical application, implantation, electroporation, lipofection, microinjection, microfluidics, biolistics, particle gun acceleration, and a combination thereof.

Illustrative embodiment 15. The method of any one of illustrative embodiments 1-14, wherein the alternating electric field is applied at a frequency in a range of from about 100 kHz to about 10 MHz.

Illustrative embodiment 16. The method of any one of illustrative embodiments 1-15, wherein the alternating electric field has a field strength of at least 1 V/cm.

Illustrative embodiment 17. The method of any one of illustrative embodiments 1-16, wherein the at least one cell is part of a biological tissue, and wherein the method further comprises the step of examining a sample of the biological tissue and identifying the target DNA sequence in the sample.

Illustrative embodiment 18. A method of treating or reducing the occurrence of at least one condition, disease, disorder, or infection in a subject, the method comprising the steps of: (1) applying an alternating electric field to at least a portion of the subject for a period of time, wherein application of the alternating electric field downregulates at least one DNA damage repair pathway in at least a portion of the subject; and (2) administering to the subject a clustered regularly interspaced short palindromic repeats (CRISPR) associated protein (CRISPR-Cas) system comprising: a single guide RNA (sgRNA) that comprises a guide sequence that hybridizes to a target DNA sequence associated with the at least one condition, disease, disorder, or infection; and a CRISPR-associated endonuclease or gene encoding same.

Illustrative embodiment 19. The method of illustrative embodiment 18, wherein the CRISPR-associated endonuclease is selected from the group consisting of Cas3, Cas9, Cas12, Cas12a, Cas12b, Cas12e, Cas12f, Cas13a, Cas13b, mini-Cas9, and Cas9 nickase (nCas9).

Illustrative embodiment 20. The method of illustrative embodiment 18 or 19, wherein the at least one condition, disease, disorder, or infection is selected from the group consisting of a cancer, an autoimmune disease, a viral infection, a bacterial infection, a fungal infection, a parasitic infection, a metabolic syndrome, Huntington's disease, Von Hippel-Lindau syndrome, and combinations thereof.

Illustrative embodiment 21. The method of illustrative embodiment 20, wherein at least one of: (a) the at least one condition, disease, disorder, or infection is a cancer, and wherein the target DNA sequence is at least one oncological gain of function gene mutation; (b) the at least one condition, disease, disorder, or infection is a cancer, and wherein the target DNA sequence is at least one loss of function mutation in a tumor suppressor gene; (c) the at least one condition, disease, disorder, or infection is a bacterial infection, and wherein the target DNA sequence is a bacterial sequence; (d) the at least one condition, disease, disorder, or infection is a viral infection, and wherein the target DNA sequence is a viral sequence; (e) the at least one condition, disease, disorder, or infection is a fungal infection, and wherein the target DNA sequence is a fungal sequence; (f) the at least one condition, disease, disorder, or infection is a parasitic infection, and wherein the target DNA sequence is a parasitic sequence; (g) the at least one condition, disease, disorder, or infection is an autoimmune disease, and wherein the target DNA sequence is a self-antigen sequence in an immune cell that produces autoantibodies; (h) the at least one condition, disease, disorder, or infection is Huntington's disease, and wherein the target DNA sequence is a CAG repeat expansion; and (i) the at least one condition, disease, disorder, or infection is a metabolic syndrome, and wherein the target DNA sequence is at least one mutation in a gene selected from the group consisting of FAH, TAT, and HPD.

Illustrative embodiment 22. The method of illustrative embodiment 21, wherein the target DNA sequence is a loss of function mutation in a tumor suppressor gene, and wherein the tumor suppressor gene is selected from the group consisting of p53, retinoblastoma (Rb), phosphatase and tensin homolog deleted on chromosome ten (PTEN), Ras association domain family (RASSF), ADP-ribosylation factor (ARF), adenomatous polyposis coli (APC), ataxia telangiectasia mutated (ATM), checkpoint kinase 2 (CHK2), breast cancer 1 protein (BRCA1), breast cancer 2 protein (BRCA2), partner and locator of BRCA2 (PALB2), tuberous sclerosis complex 1 (TSC1), tuberous sclerosis complex 2 (TSC2), neurofibromatosis type 1 (NF1), liver kinase B1 (LKB1), E2F1, KLF5, forkhead box class 03a (FOXO3a), p38, and Von Hippel-Lindau (VHL).

Illustrative embodiment 23. The method of illustrative embodiment 21, wherein the target DNA sequence is an oncological gain of function gene mutation, wherein the gene is selected from the group consisting of Ras (H-Ras, K-Ras, and N-Ras), Mitogen-activated protein kinase kinase (MEK), extracellular signal-regulated kinase (ERK), c-Jun NH2-terminal kinase (JNK), human epidermal growth factor receptor 2 (HER2), epidermal growth factor receptor (EGFR), c-Myc, fumarylacetoacetate hydrolase (FAH), TAT, 4-hydroxyphenylpyruvate dioxygenase (HPD), fms-related tyrosine kinase 3 (FLT3), c-KIT, BRAF, phosphatidylinositol-4,5-bisphosphate 3-kinase (PIK3CA), telomerase reverse transcriptase (TERT), ETS variant transcription factor 1 (ETV1), i-catenin (CTNNB1), E2F1, and T cell acute lymphocytic leukemia 1 (TAL1).

Illustrative embodiment 24. The method of illustrative embodiment 21, wherein the target DNA sequence is a sequence from a bacteria or virus selected from the group consisting of Human papillomavirus (HPV), Hepatitis B and C viruses (HBV and HCV, respectively), Epstein Barr virus (EBV), human immunodeficiency virus (HIV), SARS-CoV-2 virus, Herpes Simplex virus (HSV), Human Herpes Virus 8 (HHV-8), Human T-lymphotropic virus-1 (HTLV-1), Merkel Cell Polyomavirus (MCPyV), Cytomegalovirus (CMV), Helicobacter pylori, Chlamydia trachomatis, Salmonella typhi, Salmonella paratyphi A, Salmonella typhimurium, Streptococcus bovis, and Mycoplasma.

Illustrative embodiment 25. The method of any of illustrative embodiments 18-24, wherein the CRISPR-Cas system is administered in the form of a ribonucleoprotein (RNP) complex formed of sgRNA and endonuclease peptide or protein.

Illustrative embodiment 26. The method of any of illustrative embodiments 18-25, wherein at least one component of the CRISPR-Cas system is delivered via at least one DNA plasmid.

Illustrative embodiment 27. The method of any of illustrative embodiments 18-26, wherein at least one component of the CRISPR-Cas system is delivered via at least one DNA- or RNA-based viral vector.

Illustrative embodiment 28. The method of illustrative embodiment 27, wherein the viral vector is selected from the group consisting of an adenoviral, adeno-associated viral, lentiviral, retroviral, vaccinia, and/or herpes simplex viral vector, or an artificial virus.

Illustrative embodiment 29. The method of any of illustrative embodiments 18-28, wherein at least one component of the CRISPR-Cas system is delivered via at least one of a liposome, a lipid nanoparticle, an immunoliposome, a lipoplex, a polyplex, a poloxamer, a polycations, a virosome, a gold nanoparticle, other inorganic nanoparticle, and combinations thereof.

Illustrative embodiment 30. The method of any of illustrative embodiments 18-29, wherein at least one component of the CRISPR-Cas system is administered in naked form via straight physical delivery.

Illustrative embodiment 31. The method of any of illustrative embodiments 18-30, wherein the sgRNA and CRISPR-associated endonuclease or gene encoding same are administered by different delivery methods.

Illustrative embodiment 32. The method of any of illustrative embodiments 18-31, wherein at least one of the sgRNA and the gene encoding the CRISPR-associated endonuclease is modified to comprise a cell penetrating peptide sequence.

Illustrative embodiment 33. The method of any of illustrative embodiments 18-32, wherein at least one of the sgRNA and CRISPR-associated endonuclease or gene encoding same is administered by a route selected from the group consisting of injection, infusion, topical application, implantation, electroporation, lipofection, microinjection, microfluidics, biolistics, particle gun acceleration, and a combination thereof.

Illustrative embodiment 34. The method of any one of illustrative embodiments 18-33, wherein the CRISP-Cas system is administered about 24 hours to about 48 hours after application of the alternating electric field has begun.

Illustrative embodiment 35. The method of any one of illustrative embodiments 18-34, wherein the period of time that the alternating electric field is applied is in a range of from about 24 hours to about 72 hours.

Illustrative embodiment 36. The method of any one of illustrative embodiments 18-35, wherein the CRISP-Cas system is administered after the period of time has elapsed.

Illustrative embodiment 37. The method of any one of illustrative embodiments 18-36, wherein the alternating electric field is applied at a frequency in a range of from about 100 kHz to about 10 MHz.

Illustrative embodiment 38. The method of any one of illustrative embodiments 18-37, wherein the alternating electric field has a field strength of at least 1 V/cm.

Illustrative embodiment 39. The method of any one of illustrative embodiments 18-38, wherein steps (1) and (2) are repeated one or more times.

Illustrative embodiment 40. The method of any one of illustrative embodiments 18-39, wherein the method further comprises the step of examining a sample from the subject and identifying the target DNA sequence in the sample.

Illustrative embodiment 41. A kit, comprising: at least one pair of transducer arrays for generating an alternating electric field there between upon application of the transducer arrays to at least one cell and/or placement of the transducer arrays on a subject; and a clustered regularly interspaced short palindromic repeats (CRISPR) associated protein (CRISPR-Cas) system comprising a single guide RNA (sgRNA) that comprises: a guide sequence that hybridizes to a target DNA sequence associated with the at least one condition, disease, disorder, or infection; and a CRISPR-associated endonuclease or gene encoding same.

Illustrative embodiment 42. The kit of illustrative embodiment 41, wherein the CRISPR-associated endonuclease is selected from the group consisting of Cas3, Cas9, Cas12, Cas12a, Cas12b, Cas12e, Cas12f, Cas13a, Cas13b, mini-Cas9, and Cas9 nickase (nCas9).

Illustrative embodiment 43. The kit of illustrative embodiment 41 or 42, wherein at least one of: (a) the at least one condition, disease, disorder, or infection is a cancer, and wherein the target DNA sequence is at least one oncological gain of function gene mutation; (b) the at least one condition, disease, disorder, or infection is a cancer, and wherein the target DNA sequence is at least one loss of function mutation in a tumor suppressor gene; (c) the at least one condition, disease, disorder, or infection is a bacterial infection, and wherein the target DNA sequence is a bacterial sequence; (d) the at least one condition, disease, disorder, or infection is a viral infection, and wherein the target DNA sequence is a viral sequence; (e) the at least one condition, disease, disorder, or infection is a fungal infection, and wherein the target DNA sequence is a fungal sequence; (f) the at least one condition, disease, disorder, or infection is a parasitic infection, and wherein the target DNA sequence is a parasitic sequence; (g) the at least one condition, disease, disorder, or infection is an autoimmune disease, and wherein the target DNA sequence is a self-antigen sequence in an immune cell that produces autoantibodies; (h) the at least one condition, disease, disorder, or infection is Huntington's disease, and wherein the target DNA sequence is a CAG repeat expansion; and (i) the at least one condition, disease, disorder, or infection is a metabolic syndrome, and wherein the target DNA sequence is at least one mutation in a gene selected from the group consisting of FAH, TAT, and HPD.

Illustrative embodiment 44. The kit of illustrative embodiment 43, wherein the target DNA sequence is a loss of function mutation in a tumor suppressor gene, and wherein the tumor suppressor gene is selected from the group consisting of p53, retinoblastoma (Rb), phosphatase and tensin homolog deleted on chromosome ten (PTEN), Ras association domain family (RASSF), ADP-ribosylation factor (ARF), adenomatous polyposis coli (APC), ataxia telangiectasia mutated (ATM), checkpoint kinase 2 (CHK2), breast cancer 1 protein (BRCA1), breast cancer 2 protein (BRCA2), partner and locator of BRCA2 (PALB2), tuberous sclerosis complex 1 (TSC1), tuberous sclerosis complex 2 (TSC2), neurofibromatosis type 1 (NF1), liver kinase B1 (LKB1), E2F1, KLF5, forkhead box class 03a (FOXO3a), p38, and Von Hippel-Lindau (VHL).

Illustrative embodiment 45. The kit of illustrative embodiment 43, wherein the target DNA sequence is an oncological gain of function gene mutation, wherein the gene is selected from the group consisting of Ras (H-Ras, K-Ras, and N-Ras), Mitogen-activated protein kinase kinase (MEK), extracellular signal-regulated kinase (ERK), c-Jun NH2-terminal kinase (JNK), human epidermal growth factor receptor 2 (HER2), epidermal growth factor receptor (EGFR), c-Myc, fumarylacetoacetate hydrolase (FAH), TAT, 4-hydroxyphenylpyruvate dioxygenase (HPD), fms-related tyrosine kinase 3 (FLT3), c-KIT, BRAF, phosphatidylinositol-4,5-bisphosphate 3-kinase (PIK3CA), telomerase reverse transcriptase (TERT), ETS variant transcription factor 1 (ETV1), i-catenin (CTNNB1), E2F1, and T cell acute lymphocytic leukemia 1 (TAL1).

Illustrative embodiment 46. The kit of illustrative embodiment 43, wherein the target DNA sequence is a sequence from a bacteria or virus selected from the group consisting of Human papillomavirus (HPV), Hepatitis B and C viruses (HBV and HCV, respectively), Epstein Barr virus (EBV), human immunodeficiency virus (HIV), SARS-CoV-2 virus, Herpes Simplex virus (HSV), Human Herpes Virus 8 (HHV-8), Human T-lymphotropic virus-1 (HTLV-1), Merkel Cell Polyomavirus (MCPyV), Cytomegalovirus (CMV), Helicobacter pylori, Chlamydia trachomatis, Salmonella typhi, Salmonella paratyphi A, Salmonella typhimurium, Streptococcus bovis, and Mycoplasma.

Illustrative embodiment 47. The kit of any of illustrative embodiments 41-46, wherein the CRISPR-Cas system is in the form of a ribonucleoprotein (RNP) complex formed of sgRNA and endonuclease peptide or protein.

Illustrative embodiment 48. The kit of any of illustrative embodiments 41-47, wherein at least one component of the CRISPR-Cas system is disposed in at least one DNA plasmid.

Illustrative embodiment 49. The kit of any of illustrative embodiments 41-48, wherein at least one component of the CRISPR-Cas system is disposed in at least one DNA- or RNA-based viral vector.

Illustrative embodiment 50. The kit of illustrative embodiment 49, wherein the viral vector is selected from the group consisting of an adenoviral, adeno-associated viral, lentiviral, retroviral, vaccinia, and/or herpes simplex viral vector, or an artificial virus.

Illustrative embodiment 51. The kit of any of illustrative embodiments 41-50, wherein at least one component of the CRISPR-Cas system is disposed in at least one of a liposome, a lipid nanoparticle, an immunoliposome, a lipoplex, a polyplex, a poloxamer, a polycations, a virosome, a gold nanoparticle, other inorganic nanoparticle, and combinations thereof.

Illustrative embodiment 52. The kit of any of illustrative embodiments 41-51, wherein at least one component of the CRISPR-Cas system is provided in naked form for straight physical delivery.

Illustrative embodiment 53. The kit of any of illustrative embodiments 41-52, wherein the sgRNA and CRISPR-associated endonuclease or gene encoding same are disposed in different delivery vehicles.

Illustrative embodiment 54. The kit of any of illustrative embodiments 41-53, wherein at least one of the sgRNA and the gene encoding the CRISPR-associated endonuclease is modified to comprise a cell penetrating peptide sequence.

Illustrative embodiment 55. The kit of any of illustrative embodiments 41-54, wherein at least one of the sgRNA and CRISPR-associated endonuclease or gene encoding same is formulated for administration via a route selected from the group consisting of injection, infusion, topical application, implantation, electroporation, lipofection, microinjection, microfluidics, biolistics, particle gun acceleration, and a combination thereof.

Illustrative embodiment 56. The kit of any one of illustrative embodiments 41-55, wherein the alternating electric field is configured to be applied at a frequency in a range of from about 100 kHz to about 10 MHz.

Illustrative embodiment 57. The kit of any one of illustrative embodiments 41-56, wherein the alternating electric field is configured to be applied at a field strength of at least 1 V/cm.

Illustrative embodiment 58. The kit of any one of illustrative embodiments 41-57, further comprising at least one device and/or at least one reagent for examining a sample to identify the target DNA sequence in the sample.

Illustrative embodiment 59. A clustered regularly interspaced short palindromic repeats (CRISPR) associated protein (CRISPR-Cas) system for use in the method of any of illustrative embodiments 1-40, the CRISPR-Cas system comprising: a single guide RNA (sgRNA) that comprises a guide sequence that hybridizes to a target DNA sequence in the at least one cell; and a CRISPR-associated endonuclease or gene encoding same.

Illustrative embodiment 60. Use of a clustered regularly interspaced short palindromic repeats (CRISPR) associated protein (CRISPR-Cas) system in the method of any of illustrative embodiments 1-40, the CRISPR-Cas system comprising: a single guide RNA (sgRNA) that comprises a guide sequence that hybridizes to a target DNA sequence in the at least one cell; and a CRISPR-associated endonuclease or gene encoding same.

While the attached disclosures describe the inventive concept(s) in conjunction with the specific experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A method of inducing apoptosis in at least one cell, the method comprising the steps of:
(1) applying an alternating electric field to the at least one cell for a period of time, wherein application of the alternating electric field downregulates at least one DNA damage repair pathway in the at least one cell; and
(2) exposing the at least one cell to a clustered regularly interspaced short palindromic repeats (CRISPR) associated protein (CRISPR-Cas) system comprising:
a single guide RNA (sgRNA) that comprises a guide sequence that hybridizes to a target DNA sequence in the at least one cell; and
a CRISPR-associated endonuclease or gene encoding same.

2. The method of claim 1, wherein the CRISPR-associated endonuclease is selected from the group consisting of Cas3, Cas9, Cas12, Cas12a, Cas12b, Cas12e, Cas12f, Cas13a, Cas13b, mini-Cas9, and Cas9 nickase (nCas9).

3. The method of claim 1, wherein the target DNA sequence is selected from the group consisting of an oncological gain of function gene mutation in an oncogene, a loss of function mutation in a tumor suppressor gene, a gene of an infectious microorganism, and a self-antigen sequence in an immune cell that produces autoantibodies.

4. The method of claim 3, wherein the target DNA sequence is directed to a mutation in a gene selected from the group consisting of p53, retinoblastoma (Rb), phosphatase and tensin homolog deleted on chromosome ten (PTEN), Ras association domain family (RASSF), ADP-ribosylation factor (ARF), adenomatous polyposis coli (APC), ataxia telangiectasia mutated (ATM), checkpoint kinase 2 (CHK2), breast cancer 1 protein (BRCA1), breast cancer 2 protein (BRCA2), partner and locator of BRCA2 (PALB2), tuberous sclerosis complex 1 (TSC1), tuberous sclerosis complex 2 (TSC2), neurofibromatosis type 1(NF1), liver kinase B1(LKB1), E2F1, KLF5, forkhead box class 03a (FOXO3a), Ras (H-Ras, K-Ras, and N-Ras), Mitogen-activated protein kinase kinase (MEK), extracellular signal-regulated kinase (ERK), c-Jun NH₂-terminal kinase (JNK), p38, human epidermal growth factor receptor 2 (HER2), epidermal growth factor receptor (EGFR), c-Myc, Von Hippel-Lindau (VHL), fumarylacetoacetate hydrolase (FAH), TAT, 4-hydroxyphenylpyruvate dioxygenase (HPD), fms-related tyrosine kinase 3 (FLT3), c-KIT, BRAF, phosphatidylinositol-4,5-bisphosphate 3-kinase (PIK3CA), telomerase reverse transcriptase (TERT), ETS variant transcription factor 1 (ETV1), β-catenin (CTNNB1), E2F1, and T cell acute lymphocytic leukemia 1 (TAL1).

5. The method of claim 3, wherein the target DNA sequence is a gene from a microorganism selected from the group consisting of Human papillomavirus (HPV), Hepatitis B and C viruses (HBV and HCV, respectively), Epstein Barr virus (EBV), human immunodeficiency virus (HIV), SARS-CoV-2 virus, Herpes Simplex virus (HSV), Human Herpes Virus 8 (HHV-8), Human T-lymphotropic virus-1 (HTLV-1), Merkel Cell Polyomavirus (MCPyV), Cytomegalovirus (CMV), Helicobacter pylori, Chlamydia trachomatis, Salmonella typhi, Salmonella paratyphi A, Salmonella typhimurium, Streptococcus bovis, and Mycoplasma.

6. The method of claim 1, wherein at least one of:
the alternating electric field is applied at a frequency in a range of from about 100 kHz to about 10 MHz; and
the alternating electric field has a field strength of at least 1 V/cm.

7. The method of claim 1, wherein the at least one cell is part of a biological tissue, and wherein the method further comprises the step of examining a sample of the biological tissue and identifying the target DNA sequence in the sample.

8. A method of treating or reducing the occurrence of at least one condition, disease, disorder, or infection in a subject, the method comprising the steps of:
(1) applying an alternating electric field to at least a portion of the subject for a period of time, wherein application of the alternating electric field downregulates at least one DNA damage repair pathway in at least a portion of the subject; and
(2) administering to the subject a clustered regularly interspaced short palindromic repeats (CRISPR) associated protein (CRISPR-Cas) system comprising:
a single guide RNA (sgRNA) that comprises a guide sequence that hybridizes to a target DNA sequence associated with the at least one condition, disease, disorder, or infection; and
a CRISPR-associated endonuclease or gene encoding same.

9. The method of claim 8, wherein the CRISPR-associated endonuclease is selected from the group consisting of Cas3, Cas9, Cas12, Cas12a, Cas12b, Cas12e, Cas12f, Cas13a, Cas13b, mini-Cas9, and Cas9 nickase (nCas9).

10. The method of claim 8, wherein the at least one condition, disease, disorder, or infection is selected from the group consisting of a cancer, an autoimmune disease, a viral infection, a bacterial infection, a fungal infection, a parasitic infection, a metabolic syndrome, Huntington's disease, Von Hippel-Lindau syndrome, and combinations thereof.

11. The method of claim 10, wherein at least one of:
(a) the at least one condition, disease, disorder, or infection is a cancer, and wherein the target DNA sequence is at least one oncological gain of function gene mutation;
(b) the at least one condition, disease, disorder, or infection is a cancer, and wherein the target DNA sequence is at least one loss of function mutation in a tumor suppressor gene;
(c) the at least one condition, disease, disorder, or infection is a bacterial infection, and wherein the target DNA sequence is a bacterial sequence;
(d) the at least one condition, disease, disorder, or infection is a viral infection, and wherein the target DNA sequence is a viral sequence;
(e) the at least one condition, disease, disorder, or infection is a fungal infection, and wherein the target DNA sequence is a fungal sequence;
(f) the at least one condition, disease, disorder, or infection is a parasitic infection, and wherein the target DNA sequence is a parasitic sequence;
(g) the at least one condition, disease, disorder, or infection is an autoimmune disease, and wherein the target DNA sequence is a self-antigen sequence in an immune cell that produces autoantibodies;
(h) the at least one condition, disease, disorder, or infection is Huntington's disease, and wherein the target DNA sequence is a CAG repeat expansion; and
(i) the at least one condition, disease, disorder, or infection is a metabolic syndrome, and wherein the target DNA sequence is at least one mutation in a gene selected from the group consisting of FAH, TAT, and HPD.

12. The method of claim 11, wherein the target DNA sequence is a loss of function mutation in a tumor suppressor gene, and wherein the tumor suppressor gene is selected from the group consisting of p53, retinoblastoma (Rb), phosphatase and tensin homolog deleted on chromosome ten (PTEN), Ras association domain family (RASSF), ADP-ribosylation factor (ARF), adenomatous polyposis coli (APC), ataxia telangiectasia mutated (ATM), checkpoint kinase 2 (CHK2), breast cancer 1 protein (BRCA1), breast cancer 2 protein (BRCA2), partner and locator of BRCA2 (PALB2), tuberous sclerosis complex 1 (TSC1), tuberous sclerosis complex 2 (TSC2), neurofibromatosis type 1 (NF1), liver kinase B1 (LKB1), E2F1, KLF5, forkhead box class 03a (FOXO3a), p38, and Von Hippel-Lindau (VHL).

13. The method of claim 11, wherein the target DNA sequence is an oncological gain of function gene mutation, wherein the gene is selected from the group consisting of Ras (H-Ras, K-Ras, and N-Ras), Mitogen-activated protein kinase kinase (MEK), extracellular signal-regulated kinase (ERK), c-Jun NH₂-terminal kinase (JNK), human epidermal growth factor receptor 2 (HER2), epidermal growth factor receptor (EGFR), c-Myc, fumarylacetoacetate hydrolase (FAH), TAT, 4-hydroxyphenylpyruvate dioxygenase (HPD), fms-related tyrosine kinase 3 (FLT3), c-KIT, BRAF, phosphatidylinositol-4,5-bisphosphate 3-kinase (PIK3CA), telomerase reverse transcriptase (TERT), ETS variant transcription factor 1 (ETV1), β-catenin (CTNNB1), E2F1, and T cell acute lymphocytic leukemia 1 (TAL1).

14. The method of claim 11, wherein the target DNA sequence is a sequence from a bacteria or virus selected from the group consisting of Human papillomavirus (HPV), Hepatitis B and C viruses (HBV and HCV, respectively), Epstein Barr virus (EBV), human immunodeficiency virus (HIV), SARS-CoV-2 virus, Herpes Simplex virus (HSV), Human Herpes Virus 8 (HHV-8), Human T-lymphotropic virus-1 (HTLV-1), Merkel Cell Polyomavirus (MCPyV), Cytomegalovirus (CMV), Helicobacter pylori, Chlamydia trachomatis, Salmonella typhi, Salmonella paratyphi A, Salmonella typhimurium, Streptococcus bovis, and Mycoplasma.

15. The method of claim 8, wherein the CRISP-Cas system is administered about 24 hours to about 48 hours after application of the alternating electric field has begun.

16. The method of claim 8, wherein the CRISP-Cas system is administered after the period of time has elapsed.

17. The method of claim 8, wherein at least one of:
the alternating electric field is applied at a frequency in a range of from about 100 kHz to about 10 MHz;
the alternating electric field has a field strength of at least 1 V/cm; and
the period of time that the alternating electric field is applied is in a range of from about 24 hours to about 72 hours; and
steps (1) and (2) are repeated one or more times.

18. The method of claim 8, wherein the method further comprises the step of examining a sample from the subject and identifying the target DNA sequence in the sample.

19. A kit, comprising:
at least one pair of transducer arrays for generating an alternating electric field therebetween upon application of the transducer arrays to at least one cell and/or placement of the transducer arrays on a subject; and
a clustered regularly interspaced short palindromic repeats (CRISPR) associated protein (CRISPR-Cas) system comprising:
a single guide RNA (sgRNA) that comprises a guide sequence that hybridizes to a target DNA sequence associated with the at least one condition, disease, disorder, or infection; and
a CRISPR-associated endonuclease or gene encoding same.

20. The kit of claim 19, wherein at least one of:
(a) the at least one condition, disease, disorder, or infection is a cancer, and wherein the target DNA sequence is at least one oncological gain of function gene mutation;
(b) the at least one condition, disease, disorder, or infection is a cancer, and wherein the target DNA sequence is at least one loss of function mutation in a tumor suppressor gene;
(c) the at least one condition, disease, disorder, or infection is a bacterial infection, and wherein the target DNA sequence is a bacterial sequence;
(d) the at least one condition, disease, disorder, or infection is a viral infection, and wherein the target DNA sequence is a viral sequence;
(e) the at least one condition, disease, disorder, or infection is a fungal infection, and wherein the target DNA sequence is a fungal sequence;
(f) the at least one condition, disease, disorder, or infection is a parasitic infection, and wherein the target DNA sequence is a parasitic sequence;
(g) the at least one condition, disease, disorder, or infection is an autoimmune disease, and wherein the target DNA sequence is a self-antigen sequence in an immune cell that produces autoantibodies;
(h) the at least one condition, disease, disorder, or infection is Huntington's disease, and wherein the target DNA sequence is a CAG repeat expansion; and
(i) the at least one condition, disease, disorder, or infection is a metabolic syndrome, and wherein the target DNA sequence is at least one mutation in a gene selected from the group consisting of FAH, TAT, and HPD.
